(19) United States
(12) Patent Application Publication
(10) Pub. No.: US 2009/0010990 A1
(43) Pub. Date: Jan. 8, 2009

(54) Process for depositing calcium phosphate therapeutic coatings with controlled release rates and a prosthesis coated via the process

(76) Inventors: Marisa A. Little, Bedford, MA (US); Nader M. Kalkhoran, Tewksbury, MA (US); Arash Aslani, Acton, MA (US); Roger Little, Bedford, MA (US)

Correspondence Address: Iandiorio Teska & Coleman, 260 Bear Hill Road, Waltham, MA, 02451, US

(21) Appl. No.: 12/214,037

(22) Filed: Jun. 16, 2008

Related U.S. Application Data
(60) Provisional application No. 60/936,414, filed on Jun. 20, 2007.

(51) Int. Cl. A61F 2/28; A61L 27/32

(52) U.S. Cl. 424/423; 427/2.27

(57) ABSTRACT

A method of coating a substrate wherein the crystallinity of a calcium phosphate substance in a coating material is controlled, the calcium phosphate substance is loaded with a therapeutic agent, and the loaded calcium phosphate is deposited onto at least a portion of the substrate.

### RELATED APPLICATIONS

The subject invention claims the benefit of and priority to U.S. Provisional Application No. 60/936,414, filed Jun. 20, 2007 which is incorporated herein by reference.

### FIELD OF THE INVENTION

This subject invention relates to implants, coatings for implants, and therapeutic agents such as bone morphogenic proteins.

### BACKGROUND OF THE INVENTION

Implants made of titanium, cobalt chrome, and other materials are often coated with one or more layers of a calcium phosphate material such as hydroxyapatite to promote bony fixation (biointegration) wherein bone grows onto and/or into the surface of the implant. It is also known to add a therapeutic agent to the implant such as a bone morphogenic protein (e.g., BMP, or GDF-5) to promote bone growth.

U.S. Pat. No. 6,821,528, incorporated herein by this reference, discloses a process wherein calcium phosphate in the form of hydroxyapatite is precipitated from a solution to coat the implant. Next, the coated implant is dried, sterilized, and packaged. Just before implantation, the coated implant is immersed in a morphogenic protein solution.

In such a process, the release rate of the therapeutic agent is difficult to control. Also, the surgeon is required to immerse the implant in the morphogenic protein solution.

It is also known to adhere a hydroxyapatite layer to the surface of an implant by plasma thermal spraying. See U.S. Pat. No. 5,934,287 incorporated herein by this reference. That patent discloses a different process wherein amorphous calcium phosphate particles are sandblasted onto an implant to form a coating. A therapeutic agent is not present and thus bone growth may not be adequately promoted.

According to U.S. Pat. No. 6,949,251, also incorporated herein by this reference, an implant comprises a porous β-TCP matrix and a bioactive agent such as a bone morphogenic protein preferably encapsulated in a biodegradable agent such as a polymer. Also, a composition including β-TCP and a bioactive agent can be disposed on the surface of an implant. Polymers used in controlled delivery systems have been known to cause complications.

Patent Application No. 2006/0088565, also incorporated herein by this reference, discloses a pharmaceutical composition for bone repair wherein a calcium phosphate carrier is coated with a protein such as BMP.

U.S. Pat. No. 6,261,322 (also incorporated herein by this reference) discloses coating an implant with a biocompatible coating which may include a calcium phosphate. Physical or chemical vapor deposition is used to coat the implant. The implant may have multiple layers and/or nanolayers.

U.S. Pat. No. 6,969,474 (incorporated herein by this reference) discloses acid etching the surface of an implant and depositing particles of a bone growth enhancing material such as bone morphogenic proteins or hydroxyapatite onto the etched surface of the implant.

### SUMMARY OF THE INVENTION

It is therefore an object of this invention to provide a new method of coating an implant.

It is a further object of this invention to provide an implant which, in one example, promotes osteogenesis, osteoconduction, and osteoinduction.

It is a further object of this invention to include in the coating a therapeutic agent such as a BMP and then deposit the coating in a way in which the efficacy of the therapeutic agent is not adversely affected.

It is a further object of this invention to provide a method of tailoring the release rate of the therapeutic agent from the coating once applied to the implant and implanted into a patient.

The subject invention results from the realization that by loading calcium phosphate with a therapeutic agent such as a bone morphogenic protein and controlling the crystallinity of the calcium phosphate, the release rate of the therapeutic agent can be tailored and also that by using an Accelerated Particle Deposition (APD) process to coat the implant with the loaded calcium phosphate, the efficacy of the therapeutic agent is not adversely affected.

The subject invention, however, in other embodiments, need not achieve all these objectives and the claims hereof should not be limited to structures or methods capable of achieving these objectives.

This subject invention features a method of coating a substrate and a product made by the method. The method includes controlling the crystallinity of a calcium phosphate substance in a coating material, loading the calcium phosphate substance with a therapeutic agent, and depositing the loaded calcium phosphate onto at least a portion of the substrate. The calcium phosphate substance may be amorphous calcium phosphate, fluorapatite, hydroxyapatite, tetracalcium phosphate, tricalcium phosphate-alpha, tricalcium phosphate-beta, biphasic calcium phosphate, and/or multiphasic calcium phosphate. The calcium phosphate substance typically has a grain size between 10 nm and 10 microns.

Controlling the crystallinity may include choosing nanocrystalline calcium phosphate particles (<100 nm grain size), choosing microcrystalline calcium phosphate particles, forming powder particles of loaded calcium phosphate wherein a certain percentage of the calcium phosphate is amorphous and a certain percentage of the calcium phosphate is crystalline in structure, or forming a certain percentage of powder particles of loaded calcium phosphate having one characteristic and mixing the same with a certain percentage of powder particles of loaded calcium phosphate having a different characteristic.

Depositing may include employing a gas accelerated particle deposition process, ion beam sputtering, electrophoretic deposition, or ion beam assisted deposition. Particles of loaded calcium phosphate in one example between 0.001 to 200 μm may be entrained in a gas jet at 50 to 400 psi and directed to the surface of the substrate at a distance of 0.5 to 24 inches.

Loading may include mixing a therapeutic substance in solution with calcium phosphate in powder form. Calcium phosphate can be precipitated from a solution including the therapeutic substance. The loaded calcium phosphate can be deposited to a thickness of between 0.1-50 μm. A thickness of between 1-30 μm is preferred. In one example, the therapeutic substance is a bone morphogenic compound and/or an antibiotic.

The subject invention also features an implant with a coating on at least a portion of its surface, the coating comprising particles of calcium phosphate of a predetermined crystallinity loaded with a therapeutic agent imbedded into the implant. The therapeutic compound is released from the coating in a controlled, predetermined manner.

The calcium phosphate substance may be amorphous calcium phosphate, fluorapatite, hydroxyapatite, tetracalcium phosphate, tricalcium phosphate-alpha, tricalcium phosphate-beta, biphasic calcium phosphate, and/or multiphasic calcium phosphate. The calcium phosphate grain size may be between 10 μm and 100 microns. Grain sizes between 10 nm and 100 microns are preferred. The calcium phosphate may include nanocrystalline calcium phosphate particles. The calcium phosphate may also include microcrystalline calcium phosphate particles. A certain percentage of the calcium phosphate may be amorphous and a certain percentage of the calcium phosphate may be crystalline in structure. A certain percentage of powder particles of loaded calcium phosphate may have one characteristic and can be mixed with a certain percentage of powder particles of loaded calcium phosphate having a different characteristic. A gas accelerated particle deposition process, ion beam sputtering, electrophoretic deposition, or ion beam assisted deposition can be used to coat the particles onto the implant. Particles of loaded calcium phosphate between 0.01 to 200 μm may be entrained in a gas jet at 50 to 220 psi and directed to the surface of an implant at a distance of 0.5 to 24 inches.

A therapeutic substance in solution may be mixed with calcium phosphate in powder form. Calcium phosphate can be precipitated from a solution including the therapeutic substance. A typical coating has a thickness of between 0.1-50 μm. The therapeutic substance may be a bone morphogenic compound, an antibiotic, an anti-inflammatory agent, an anti-microbial agent, and/or stem cells.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a block diagram showing the primary components associated with an exemplary subsystem used to deposit a calcium phosphate coating onto a substrate in accordance with the subject invention;

FIG. 2 is a SEM cross-sectional view of a calcium phosphate coating applied to a titanium substrate in accordance with the subject invention;

FIG. 3 is another SEM cross-sectional view of a calcium phosphate coating applied to a titanium substrate in accordance with the subject invention;

FIG. 4 is a profilometry scan of an uncoated substrate;

FIG. 5 is a profilometry scan of a substrate coated with calcium phosphate in accordance with the subject invention;

FIG. 6 is a graph showing the tensile strength for various calcium phosphate coatings in accordance with the subject invention;

FIG. 7 is a graph showing the shear strength of a variety of calcium phosphate coatings;

FIG. 8 is a graph showing the release profile of a therapeutic agent in micrograms over time when the coating includes amorphous calcium phosphate in accordance with the subject invention;

FIG. 9 is a graph showing the release profile of a therapeutic agent in micrograms over time when the coating includes microcrystalline calcium phosphate in accordance with the subject invention;

FIG. 10 is a graph showing the percent change in the osteoblast number when cultured with supernatant of BMP-2 released from a nano-amorphous calcium phosphate coating over 21 days; and

FIG. 11 is a highly schematic cross-sectional view of an example of a calcium phosphate particle loaded with a therapeutic agent in accordance with the subject invention.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

In accordance with the method of the subject invention, a substrate is coated with calcium phosphate loaded with a therapeutic agent. The crystallinity of the calcium phosphate is controlled to control the release rate of the therapeutic agent. Typically, the loaded calcium phosphate is deposited onto the substrate by an APD process. Ion beam sputtering, electrophoretic deposition, ion beam assisted deposition, and other methods, however, may be used. Typically a therapeutic substance such as a bone morphogenic protein is mixed in solution with calcium phosphate in powder form. Or, the calcium phosphate can be precipitated from a solution including the therapeutic substance.

A typical thickness of the loaded calcium phosphate coating is between 0.1 to 50 μm. In one example, when an APD process is used, particles of loaded calcium phosphate between 0.001 to 200 μm are entrained in a gas jet at 50-220 psi and then directed to the surface of an implant at a distance of typically 0.5-24 inches.

The subject invention represents a process for deposition of a coating including calcium phosphate (CaP) which has been loaded with one or more therapeutic substances. The coating is engineered to release the embedded substance at a controlled rate usually for a local therapeutic effect. The deposition process preferably occurs at room temperature which allows the source material to maintain its original size, chemistry and phase composition. Inert gas, such as nitrogen, may be used in the application of the coating but is not incorporated into the coating. In other words, the composition of the source material is exactly what ends up in the coating. Deposition typically occurs at room temperature or lower (<100° C.) so the therapeutic agent is not adversely affected. The lower temperatures also assist in controlling the crystallinity of the calcium phosphate material. Typical temperatures are between 100° C. and −196° C.

Various nozzle tips have been tested and found to variably affect the APD process and resulting coating properties. The CaP particles are accelerated to such a speed that when they impact the substrate, the particles imbed themselves into the substrate and form a layer of CaP. The coating's adherence and coherence depends on the source material, the substrate material, and various processing parameters, such as pressure, the angle of incidence and the distance from the nozzle to the sample. See also U.S. Pat. Nos. 5,302,414; 6,502,767; 4,968,540 and Application Publication No. 2005/0169964A1 all incorporated herein by this reference.

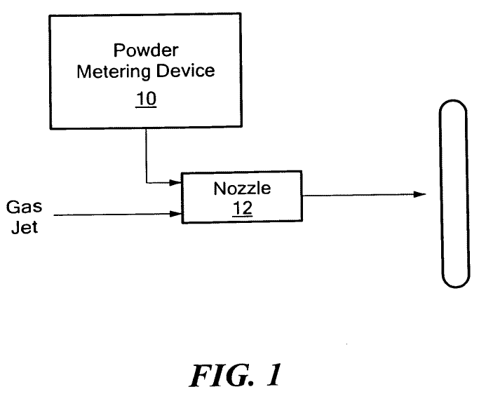

The coating media (CaP powder) is placed in a reservoir 10, FIG. 1 which is then sealed and connected to a control unit. Deposition takes place inside a glovebox, which is under negative air pressure to prevent CaP from escaping into the room. CaP powder is drawn through the control unit and exits through nozzle 12 at a very high acceleration rate. The pressure and deposition rate can be controlled. The deposition stream can also be controlled by the size and geometry of the nozzle, the distance from nozzle to the substrate, and the deposition angle. Relative motion can be provided between the nozzle and the substrate.

The therapeutic substance, e.g., drug or protein, is incorporated into the source material prior to the coating process to promote even distribution of the therapeutic substance throughout the coating. The distribution of the drug(s) have an influence on the release kinetics of the coating.

Another influence on the release kinetics is the crystalline composition of the calcium phosphate. By controlling the crystallinity of the source material and coating, the dissolution rate of the calcium phosphate (scaffold holding the drug in place) can be controlled. Phase compositions that could be utilized in this coating are 100% crystalline hydroxyapatite (HA), highly crystalline HA (80-90%), tricalcium phosphate (TCP), beta tricalcium phosphate (β-TCP), and amorphous calcium phosphate (ACP). The coating could also be composed of one or more of these calcium phosphate phases. The various phases could be applied all at once or they could be deposited in distinct layers to form a graded coating. The graded coating would provide a method for further controlling the coating response to the environment and the body's response to the coating and implant.

A calcium phosphate-based coating with a therapeutic substance incorporated is advantageous because most drug-eluting coatings on the market today are polymer based. Polymers can elicit an inflammatory response from the body whereas calcium phosphate is a naturally occurring substance in the body and would therefore prevent any foreign body response.

The new drug-eluting coatings can be used for a variety of implanted medical devices and applications including cardiovascular stents and bone-contacting implants.

### EXAMPLE

The APD process can accelerate particles to subsonic or supersonic speeds. Using this process, particles have been deposited on a variety of substrates including Ti, SS, CoCr, and the like. The substrate may be metal, a polymer, or a ceramic either with or without an existing coating.

The process gas is introduced through a gas control module to a manifold system containing Nitrogen gas to a powder-metering device. The high-pressure gas is introduced into the nozzle; the gas accelerates to sonic velocity in the throat region of the nozzle. The flow then becomes supersonic as it expands in the diverging section of the nozzle. See FIG. 1. Typical gas-jet parameters for the process are summarized in Table 1:

TABLE 1

| Parameter | Range |
|---|---|
| Operation Gas | Air, nitrogen, helium and mixture |
| Jet Internal Pressure | 50 to 400 psi (50-200 psi preferred) |
| Jet Temperature | 20 to 30° C. |
| Spray Distance | 0.5 to 24 inches |
| | (0.5-2 inches preferred) |
| Particle size | .001 to 100 μm |

As shown in Table 1, process gases include nitrogen, helium, air, and mixtures of these gases. Nitrogen is a favored process gas because it can be used to spray some materials without promoting oxidation.

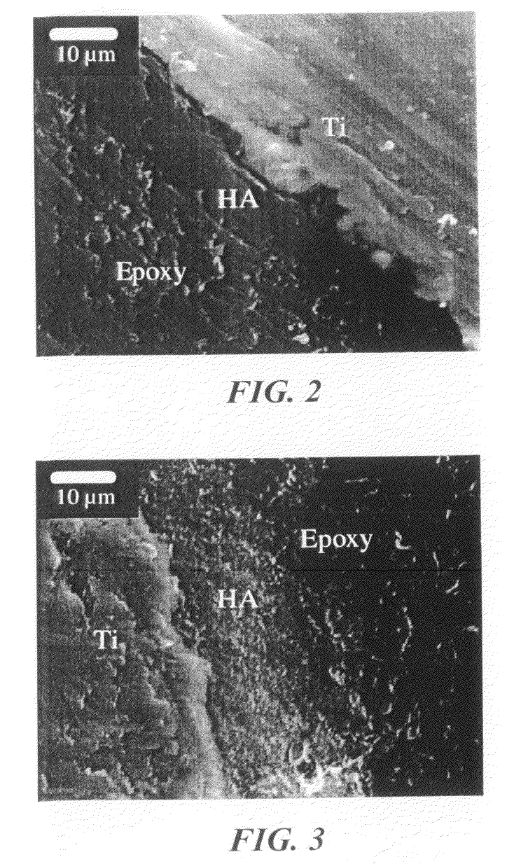

The coating thickness of one coated titanium sample was approximately 10 μm. (See FIGS. 2-3). The l.c.epoxy shown is used in shear and tensile strength testing.

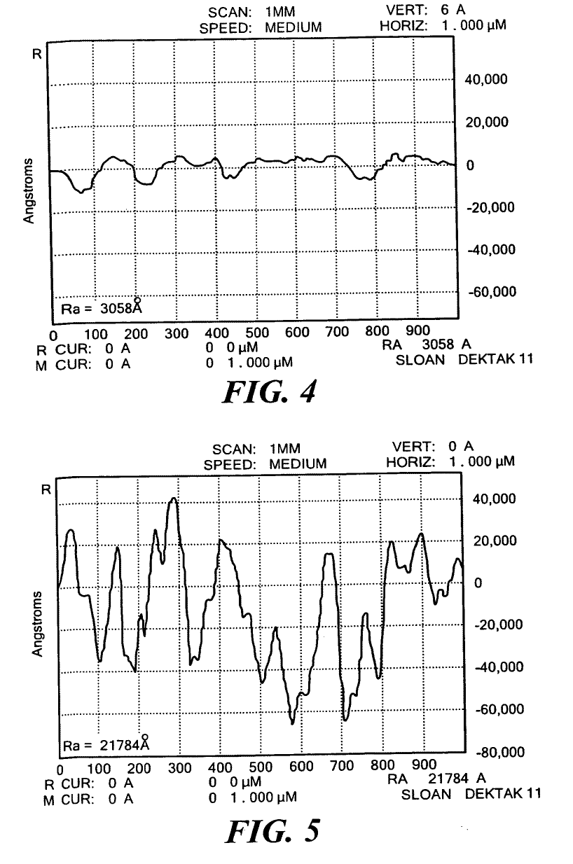

Surface roughness was evaluated for a HA coating deposited on polished titanium samples. Results of surface profilometry evaluations are shown in FIGS. 4-5. FIG. 4 is a profilometry scan of uncoated substrates and FIG. 5 is a scan of a coated substrate. The X-axis units are micrometers; the Y-axis units are Angstroms. The position of origin on both axes is arbitrary. As shown, the surface roughness increases from 0.3 to 2.2 microns Rₐ. The increased roughness is primarily a consequence of the HA coating, and not increased surface roughness induced by the physical bombardment process. Separate experiments demonstrated that stripping the coating away with hydrochloric acid yields the same, pre-coated roughness levels of approximately 0.3 microns.

The coating bond strength was measured according to ASTM Standard F 1147-99, “Standard Test Method for Tension Testing of Calcium Phosphate and Metallic Coatings.” The substrate was Ti-6Al-4V coupons with a dimension of 1.0 inch in diameter and 0.25 inch in thickness. The face of each uncoated coupon was bead blasted with #30 alumina granules before each bond strength test. The adhesive used with the calcium phosphate coating was FM 1000 having a thickness of 0.25 mm. A constant load was applied between the HA coated specimen and the opposition coupon, using a calibrated high temperature spring to apply a stress of 0.138 MPa, (20 psig) during the 2 to 3-hour curing process at 176° C. The bond strength test was performed using a standard tensile test machine with a constant crosshead speed of 0.25 cm/min. The fracture load and fracture surface was recorded for six samples of each HA coating composition to obtain average bond strength and standard deviation. These results were compared to commercially available plasma sprayed HA coatings.

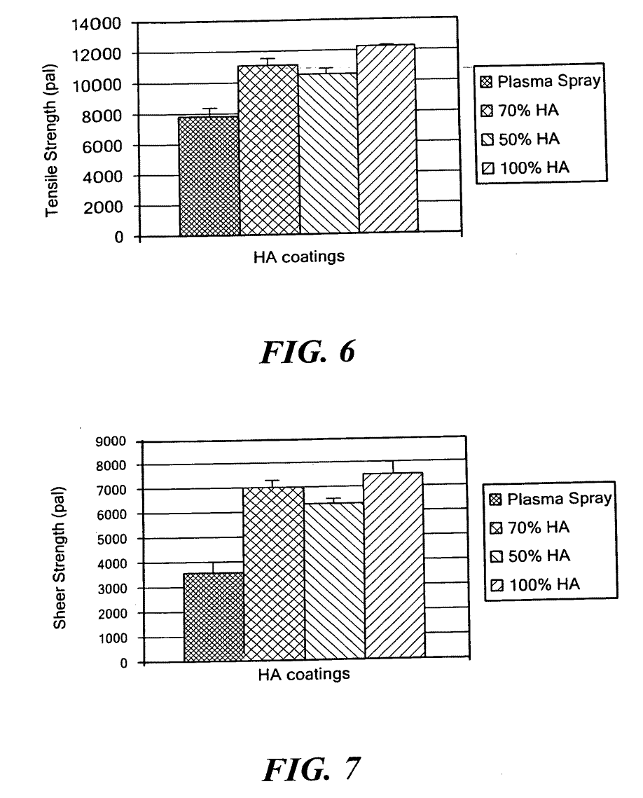

FIG. 6 shows the tensile strength on the different HA coatings per the ASTM standard compared to plasma sprayed HA. These results show that coatings applied in accordance with the process discussed above have better adhesion compared to available commercial plasma sprayed HA.

The shear bond strength of the coating-Ti interface was measured following ASTM F 1658-95, “Standard Test Method for Shear Testing of Calcium Phosphate Coatings.” FM 1000 Adhesive Film (American Cyanamid, N.J.) (with a thickness of 0.25 mm) was used. Six coated Ti6Al4V specimens (cross sectional area of 2.84 cm²) from each HA treatment type were compared to uncoated Ti alloy samples. The bond was achieved at 176° C. for 2-3 hours and at a constant stress of 0.138 MPa using a calibrated high temperature spring. The cured samples were then tested using an Instron pull tester, at a uniform cross-head speed of 0.25 cm/min. The shear strength was calculated using the following formula:

S=Pₘₐₓ/A, (1)

where S is the shear strength (MPa), Pₘₐₓ is the maximum load in the test (N), and A is the cross sectional area of the bonded area (cm²).

These results were compared to commercially available plasma sprayed HA coatings. FIG. 7 shows the shear strength of the different HA coatings per the ASTM standard compared to plasma sprayed HA. The results show that the inventive coatings have better adhesion compared to available commercial plasma sprayed HA coatings.

Thus, the preferred coating has tensile strength greater than 8000 psi, a shear strength greater than 5000 psi, and a thickness of 1-20 μm.

To produce the coating, in one example, a therapeutic substance in solution is mixed with the calcium phosphate powder prior to deposition. Therefore, the therapeutic compound is present at all levels of the coating, providing another parameter for controlling the release of the compound.

Another method for incorporating the therapeutic substance into the calcium phosphate (CaP) material is to precipitate the calcium phosphate from a solution containing the therapeutic substance. By using this method, the therapeutic substance becomes embedded within calcium phosphate agglomerates. Additionally the therapeutic substance adsorbs to the surface of the calcium phosphate agglomerates. This method may be used for materials that are prepared at a temperature less than 100° C.

Experiments were conducted to test the release rate of BMP from various formulations of calcium phosphate. Results have shown that the release rate is dependent on the crystallinity of the calcium phosphate material. Various formulations of calcium phosphate with BMP were coated on commercially pure (CP) titanium (Ti) coupons (1 cm×1 cm) using the method described above. To measure the release rate, the coated substrates were placed in 12 well plates with cell culture medium (DMEM supplemented with 10% FBS (does not contain BMP); Hyclone) and cultured at 37° C. in 95/5% air/CO₂ for up to 21 days. Once a day for 21 days, a small amount (5 microliters) of the supernatant solution was removed from each well and the presence of the imbedded proteins were determined by an ELISA assay with antibodies specific to BMP (Biochem). In this manner, the release rates of BMP per each substrate coating type were determined. Experiments were run in triplicate and repeated at three different times.

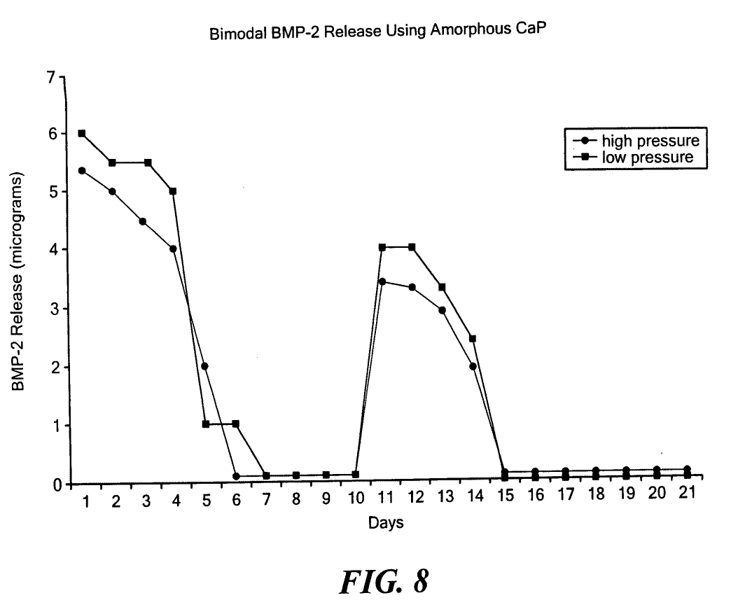

It was found that nano-amorphous CaP with BMP exhibited a bimodal release profile as seen in FIG. 8. FIG. 8 shows the BMP-2 release profile in micrograms over time using amorphous CaP and shows a bimodal release from day 1 to day 7 and day 10 to day 15. The different lines designate different pressures used during the deposition process. There is an immediate release of BMP from the coating that takes place until days 5 or 6. There is no release of BMP from day 6 or 7 to day 10. There is another release of BMP during days 11-15. Finally, there is no BMP present in the solution from day 15 until the end of the testing at day 21. The two different lines in the graph designate two different pressures that were used to deposit the coating, low and high pressure. It was found that the pressure used for deposition affected the density and release rates of the coatings and was therefore used as another parameter to control release rates of the coatings.

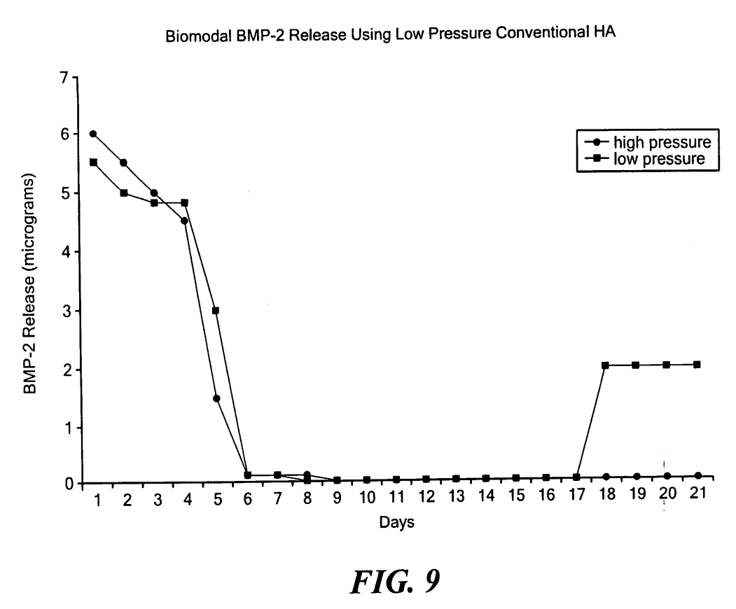

In another example, microcrystalline HA was loaded with BMP and then coated onto CPTi substrates. These samples were analyzed as described above for release rate. FIG. 9 shows BMP-2 release profile in micrograms over time using microcrystalline HA, exhibiting a bimodal release from day 1 to day 8 and day 17 through day 21 (end of assay). The coating was still present at the end of the assay so BMP release could continue for an unknown period of time. The different lines designate different pressures used during the deposition process. The bimodal release profile shown here is similar to the nano-amorphous CaP/BMP coating. However, one difference is the timing of the second release. The microcrystalline HA/BMP coating releases BMP from day 17 through the end of the test at 21 days.

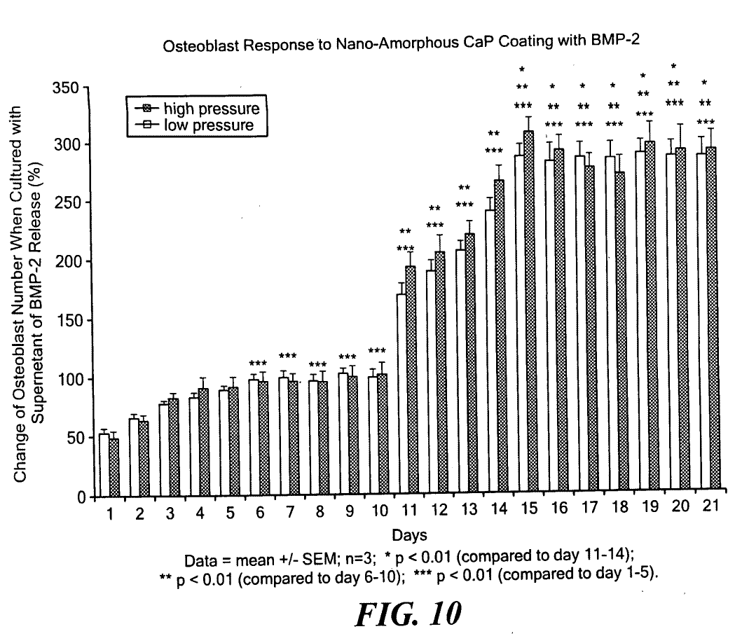

In order to verify that the BMP-2 remained active after the deposition process, coated and uncoated samples were analyzed for cell activity. It is known that BMP causes osteoblasts to proliferate and induces bone formation. To evaluate cell proliferation, human osteoblasts were seeded (3500 cell/cm²) onto glass and were cultured in the presence of the supernatant of the coatings placed in cell culture media for up to 21 days. Cell preparations were examined using a fluorescence microscope with cell density (cells per unit surface area) determined by averaging the number of cells in five random fields. Results of these cell counts were compared to controls (osteoblasts cultured without any supernatant). FIG. 10 shows the percent change in osteoblast number when cultured with supernatant of BMP-2 that has been released from nano-amorphous CaP coating over 21 days. The sharp increase in osteoblast number at day 11 directly correlates with the second release of BMP-2 from the nano-amorphous CaP coating.

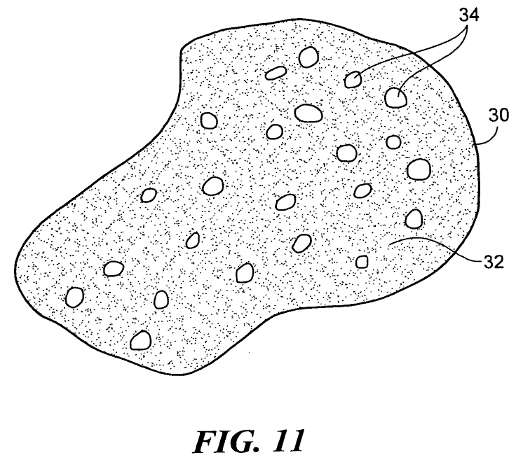

FIG. 11 shows a calcium phosphate particle 30 loaded with a therapeutic substance 32 amongst calcium phosphate powder grains 34 in accordance with the subject invention. The calcium phosphate powder grains may be amorphous calcium phosphate, fluorapatite, hydroxyapatite, tetracalcium phosphate, tricalcium phosphate-alpha, tricalcium phosphate-beta, biphasic calcium phosphate, and/or multiphasic calcium phosphate. The therapeutic substance 32 may be a bone morphogenic compound, an antibiotic, or another therapeutic substance or a combination of substances as are known in the art. The volume by weight of calcium phosphate to the therapeutic substance may be equal or of other percentages. The typical calcium phosphate grain 34 is between 10 nm and 10 microns. The crystallinity of the calcium phosphate grains can change. In one example, the agglomerated grains of hydroxyapatite are nano-crystalline in structure. In another example, they are micro-crystalline in structure. A certain percentage of particle 30 can include calcium phosphate in an amorphous state and a certain percentage of calcium phosphate in a crystalline state. Or, certain loaded particles could vary such that a certain percentage of the powder particles of loaded calcium phosphate have one characteristic (nanocrystalline in structure, for example) and they are mixed with a certain percentage of powder particles 30 of loaded calcium phosphate having different characteristics (amorphous calcium phosphate, for example).

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant can not be expected to describe certain insubstantial substitutes for any claim element amended.

What is claimed is:

1. A method of coating a substrate, the method comprising:
controlling the crystallinity of a calcium phosphate substance in a coating material;
loading the calcium phosphate substance with a therapeutic agent; and
depositing the loaded calcium phosphate onto at least a portion of the substrate.

2. The method of claim 1 in which the calcium phosphate substance is amorphous calcium phosphate, fluorapatite, hydroxyapatite, tetracalcium phosphate, tricalcium phosphate-alpha, tricalcium phosphate-beta, biphasic calcium phosphate, and/or multiphasic calcium phosphate.

3. The method of claim 1 in which the calcium phosphate substance has a grain size of between 10 nm and 100 microns.

4. The method of claim 1 in which the calcium phosphate substance has a grain size between 10 nm and 10 microns.

5. The method of claim 1 in which controlling the crystallinity includes choosing nanocrystalline calcium phosphate particles.

6. The method of claim 1 in which controlling the crystallinity includes choosing microcrystalline calcium phosphate particles.

7. The method of claim 1 in which controlling the crystallinity includes forming powder particles of loaded calcium phosphate wherein a certain percentage of the calcium phosphate is amorphous and a certain percentage of the calcium phosphate is crystalline in structure.

8. The method of claim 1 in which controlling the crystallinity includes forming a certain percentage of powder particles of loaded calcium phosphate having one characteristic and mixing the same with a certain percentage of powder particles of loaded calcium phosphate having a different characteristic.

9. The method of claim 1 in which depositing includes employing a gas accelerated particle deposition process, ion beam sputtering, electrophoretic deposition, or ion beam assisted deposition.

10. The method of claim 1 in which depositing includes employing a process wherein particles of loaded calcium phosphate between 0.001 to 200 μm are entrained in a gas jet at 50 to 400 psi and directed to the surface of the substrate at a distance of 0.5 to 24 inches.

11. The method of claim 1 in which loading includes mixing a therapeutic substance in solution with calcium phosphate in powder form.

12. The method of claim 1 in which loading includes precipitating calcium phosphate from a solution including the therapeutic substance.

13. The method of claim 1 in which depositing includes depositing the loaded calcium phosphate to a thickness of between 0.1-50 μm.

14. The method of claim 1 in which the therapeutic substance is a bone morphogenic compound, an antibiotic, an anti-inflammatory agent, an anti-microbial agent, peptide, protein and/or stem cells.

15. The method of claim 1 in which controlling the crystallinity of the calcium phosphate substance includes maintaining the temperature of the calcium phosphate substance and the therapeutic agent and depositing the same at a temperature less than 100° C.

16. An implant with a coating on at least a portion of its surface, the coating comprising particles of calcium phosphate of a predetermined crystallinity loaded with a therapeutic agent imbedded into the implant's surface, the therapeutic compound released from the coating in a controlled, predetermined manner.

17. The implant of claim 16 in which the calcium phosphate substance is amorphous calcium phosphate, fluorapatite, hydroxyapatite, tetracalcium phosphate, tricalcium phosphate-alpha, tricalcium phosphate-beta, biphasic calcium phosphate, and/or multiphasic calcium phosphate.

18. The implant of claim 16 in which the calcium phosphate has a grain size of between 10 nm and 100 microns.

19. The implant of claim 16 in which the calcium phosphate has a grain size between 10 nm and 10 microns.

20. The implant of claim 16 in which the calcium phosphate includes nanocrystalline calcium phosphate particles.

21. The implant of claim 16 in which the calcium phosphate includes microcrystalline calcium phosphate particles.

22. The implant of claim 16 in which a certain percentage of the calcium phosphate is amorphous and a certain percentage of the calcium phosphate is crystalline in structure.

23. The implant of claim 16 in which a certain percentage of powder particles of loaded calcium phosphate have one characteristic and are mixed with a certain percentage of powder particles of loaded calcium phosphate having a different characteristic.

24. The implant of claim 16 in which a gas accelerated particle deposition process, ion beam sputtering, electrophoretic deposition, or ion beam assisted deposition is used to coat the particles onto the implant.

25. The implant of claim 16 in which particles of loaded calcium phosphate between 0.001 to 200 μm are entrained in a gas jet at 50 to 400 psi and directed to the surface of an implant at a distance of 0.5 to 24 inches.

26. The implant of claim 16 in which a therapeutic substance in solution is mixed with calcium phosphate in powder form.

27. The implant of claim 16 in which calcium phosphate is precipitated from a solution including the therapeutic substance.

28. The implant of claim 16 in which the coating has a thickness of between 0.1-50 μm.

29. The implant of claim 16 in which the therapeutic substance is a bone morphogenic compound, an antibiotic, an anti-inflammatory agent, an anti-microbial agent, and/or stem cells.

30. A product made by the process of claim 1.
